(19) United States
(12) Patent Application Publication
(10) Pub. No.: US 2025/0134668 A1
(43) Pub. Date: May 1, 2025

(54) AUGMENT FOR A BONE IMPLANT, SET OF AUGMENTS

(71) Applicant: Aesculap AG, Tuttlingen (DE)

(72) Inventor: Martin Hirt, Stockach (DE)

(21) Appl. No.: 18/925,237

(22) Filed: Oct. 24, 2024

(30) Foreign Application Priority Data
Oct. 27, 2023 (DE) 10 2023 129 788.0

(51) Int. Cl. A61F 2/30; A61F 2/38

(52) U.S. Cl. CPC A61F 2/30734; A61F 2002/30326; A61F 2002/30736; A61F 2/3859; A61F 2/389

(57) ABSTRACT

An augment for a bone implant, in particular for a tibia or a femur, has a body. The body has an interface for fastening the augment to a plateau of the bone implant, an interface for fastening the augment to the bone, and an interface to a fastening element for anchoring the bone implant in a medullary cavity of a bone, which extends between the interface for fastening the augment to the plateau and the interface for fastening the augment to the bone. The body has an undercut at the interface to the fastening element. The augment can be part of a set of augments.

### CROSS-REFERENCE TO RELATED APPLICATION

This application claims priority under 35 U.S.C. § 119 to German Application No. 10 2023 129 788.0, filed on Oct. 27, 2023, the content of which is incorporated by reference herein in its entirety.

### FIELD

The present disclosure relates generally to bone implants, and more particularly to an augment for a bone implant and a set of such augments. The augment achieves improved cement fixation compared to conventional augments.

### SUMMARY

An augment for a bone implant, in particular for a tibia or a femur, has a body. The body has an interface for fastening the augment to a plateau of the bone implant, an interface for fastening the augment to the bone, and an interface to a fastening element for anchoring the bone implant in a medullary cavity of a bone, in particular of the tibia or the femur, which extends between the interface for fastening the augment to the plateau and the interface for fastening the augment to the bone. The body has an undercut at the interface to the fastening element. Bone cement in the undercut improves cement fixation.

It may be provided that the undercut comprises a conical or concave course of the body from the interface for fastening the augment to the bone to the interface for fastening the augment to the plateau. This makes it easier to manufacture the augment.

It may be provided that the body extends between the interface for fastening the augment to the plateau and the interface for fastening the augment to the bone at a first height, wherein the undercut extends between the interface for fastening the augment to the plateau and the interface for fastening the augment to the bone at a second height which is smaller than the first height, in particular from the second height to the interface for fastening the augment to the plateau.

In one embodiment, the interface for fastening the augment to the bone extends substantially in a plane, wherein the undercut comprises a wall of the body which is arranged at least in sections at an angle between 89° and 45°, in particular between 89° and 79°, to the plane.

It may be provided that the body tapers outside the interface to the fastening element from the interface for fastening the augment to the plateau to the interface for fastening the augment to the bone, or that the interface for fastening the augment to the bone extends substantially in the plane, wherein the body extends outside the interface to the fastening element from the interface for fastening the augment to the plateau to the interface for fastening the augment to the bone at an angle of 90° or substantially 90° to the plane. This means that the undercut is not arranged on surfaces of the body that are not used for cement fixation.

It may be provided that the body tapers from the interface for fastening the augment to the plateau to the interface for fastening the augment to the bone.

The body may include a circumferential area between the interface for fastening the augment to the plateau and the interface for fastening the augment to the bone, wherein the area comprises the interface to the fastening element. The undercut is integrated into the area, for example.

For example, the area has a rough surface at least in sections at the interface to the fastening element. This enables improved cement fixation.

For example, the area has a smooth surface outside the interface to the fastening element. This means that no rough surface is provided in areas unused for cement fixation.

In one embodiment, the interface to the fastening element comprises a portion of a circumference of the body extending between the interface for fastening the augment to the plateau and the interface for fastening the augment to the bone, wherein the undercut extends along the portion of the circumference, in particular over the entire portion of the circumference. This enables improved cement fixation.

A set of augments can be provided which comprises at least two of the augments, wherein the body of the at least two augments extends between the interface for fastening the respective augment to the plateau and the interface for fastening the augment to the bone at different heights, or the interface to the fastening element is designed for fastening elements of different sizes, or the undercuts differ in their shape or angle.

It may be provided that the set of augments comprises a first augment and a second augment, wherein the first augment and the second augment can be arranged against each other, wherein the interface for fastening the first augment to the plateau of the bone implant and the interface for fastening the second augment to the bone are designed to connect the first augment and the second augment.

It is possible that the second augment in the set of augments is designed without an undercut at its interface with the fastening element. This means that the second augment can be a conventional augment.

### BRIEF DESCRIPTION OF THE DRAWINGS

Further advantageous embodiments can be found in the following descriptions and the drawings, of which:

FIG. 1a shows a schematic, perspective view of an augment for a bone implant for a tibia;

FIG. 1b shows a top view of the augment for the bone implant for the tibia;

FIG. 1c shows a section through the augment for the bone implant for the tibia;

FIG. 2 shows a schematic, perspective view of the augment and at least a portion of the bone implant for the tibia;

FIG. 3a shows a top view of an augment for a bone implant for a femur;

FIG. 3b shows a section through the augment for the bone implant for the femur;

FIG. 4 shows a schematic, perspective view of the augment for the bone implant for the femur; and

FIG. 5 shows a schematic, perspective view of the augment and at least a portion of the bone implant for the femur.

### DETAILED DESCRIPTION

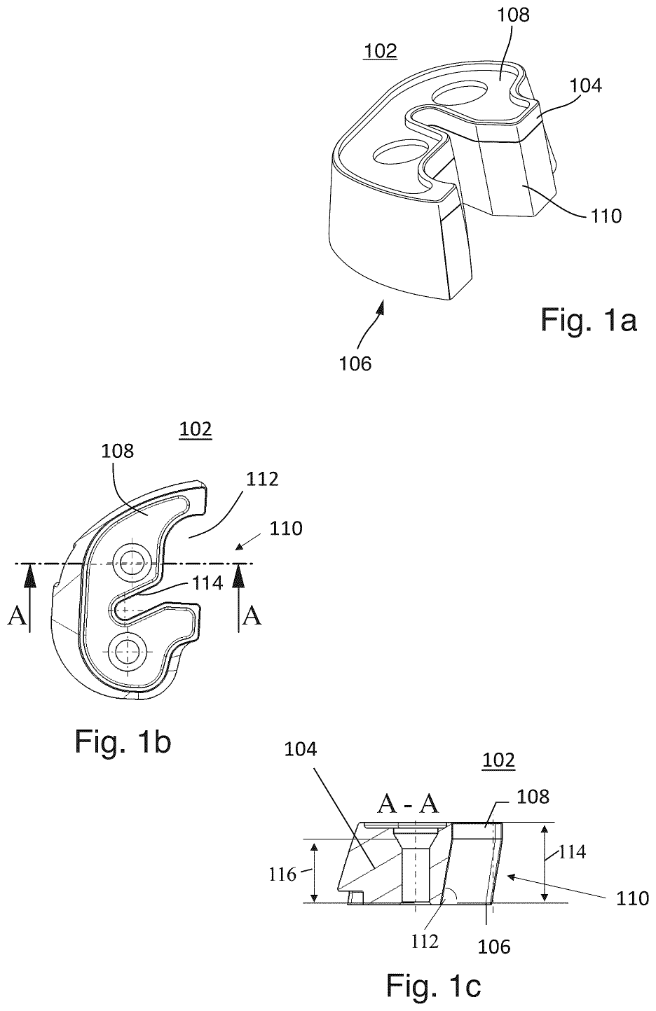

FIG. 1a shows a schematic, perspective view of an augment 102 for a bone implant for a tibia.

The augment 102 has a body 104. The body 104 has a first interface 106. The first interface 106 is designed for fastening the augment 102 to a plateau of the bone implant. The body 104 has a second interface 108. The second interface 108 is designed for fastening the augment 102 to the bone. The body 104 has a third interface 110 to a fastening element for anchoring the bone implant in a medullary cavity of a bone.

The third interface 110 extends between the first interface 106 and the second interface 108.

The body 104 includes a circumferential area between the first interface 106 and the second interface 108. The area comprises the third interface 110.

The area has a rough surface at least in sections at the third interface 110. The surface at the third interface 110 has a mean roughness value greater than 1.5 μm, particularly in the range from 3.5 μm to 9 μm. As a result, a bone cement will adhere securely to the rough surface of the third interface 110. By way of comparison, a smaller mean roughness value, e.g. a mean roughness value Ra≤1.5 μm, indicates a smooth surface.

The surface outside third interface 110 may be provided with a smooth surface.

The body 104 has an undercut at the third interface 110.

The third interface 110 comprises a portion of a circumference of the body 104 extending between the first interface 106 and the second interface 108. In the example, the undercut extends along the portion of the circumference, in particular over the entire portion of the circumference.

FIG. 1b shows a top view of the augment 102 for the bone implant for the tibia. The third interface 110 includes a first protrusion 112 having a shape corresponding to a shape of a sidewall of a tibial bone implant fastening element in the region of the third interface 110. The third interface 110 has a second protrusion 114 in the first protrusion 112, which has a shape corresponding to a shape of a sidewall of a wing of the bone implant for the tibia in the region of the third interface 110.

FIG. 1c shows a section A-A through the augment 102 for the bone implant for the tibia at a section line labeled A-A in FIG. 1b.

In the example, the second interface 108 extends substantially in one plane. In the example, the undercut comprises a wall of the body 104, which is arranged at least in sections at an angle between 89° and 45°, in particular between 89° and 79°, to the plane.

In one example, the undercut has a conical or concave shape of the body 104 from the second interface 108 to the first interface 106.

The body 104 extends between the first interface 106 and the second interface 108 at a first height 114. In the example, the undercut extends between the first interface 106 and the second interface 108 at a second height 116. The second height 116 is smaller than the first height 114. In the example, the second height 116 is a fraction, in particular at least one sixth or at least one fifth or at least one fourth, of the first height 114.

In the example, the undercut extends from the second height 116 to the first interface 106.

In the example, the body 104 tapers outside the third interface 110 from the first interface 106 to the second interface 108. In the example, the body 104 tapers from the first interface 106 to the second interface 108. It may be provided that the body 104 extends outside the third interface 110 from the first interface 106 to the second interface 108 at an angle of 90° to the plane. It may be provided that the body 104 tapers only outside the third interface 110.

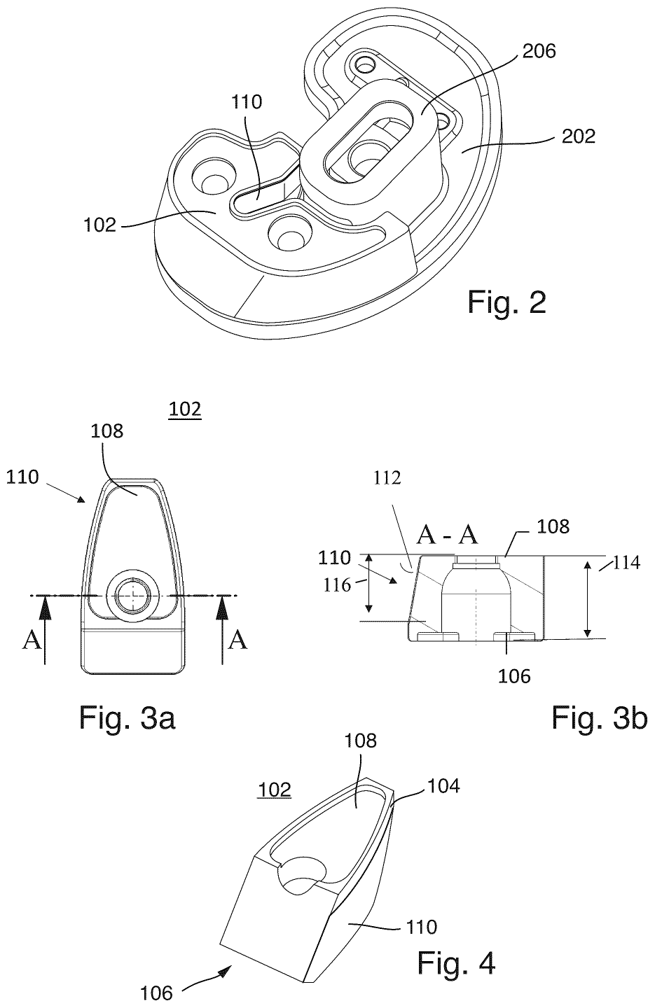

FIG. 2 shows a schematic, perspective view of the augment 102 and at least part of the bone implant for the tibia.

The bone implant comprises a plateau 202 that can be positioned on a tibia that has been prepared with a horizontal saw cut for this purpose. The bone implant has a fastening element 206, which is designed for anchoring the bone implant in a medullary cavity of the tibia prepared for this purpose.

The third interface 110 is designed to receive the portion of the fastening element 206. Fastening element 206 comprises a coupling element with a central axis. In the example, the coupling element is an opening into which a shaft, which is not shown in detail in the drawing, is inserted, in particular by screwing. In this case, the opening is a threaded hole. The central axis of this threaded hole, i.e. of the coupling element, is, for example, perpendicular or substantially perpendicular to the plateau 202. In the example, the third interface 110 and the portion of the fastening element 206 have corresponding area shapes and sizes. For example, between the third interface 110 and the fastening element 206 there is a gap-shaped cement pocket, the base of which forms the plateau 202. The cement pocket is open towards the bone, in this case the tibia. Bone cement can therefore enter the cement pocket through an opening formed there and, after hardening, wedge itself into the cement pocket. For this wedging, the undercut is provided at the third interface 110, the surface of which moves radially further and further away from the extended axis of the threaded hole in the direction of the plateau 202. As a result, it can be provided that the surface of the third interface 110 moves further and further away from the extended axis of the coupling element over its height 116 in the direction of the plateau 202.

FIG. 3a shows a top view of an augment 102 for a bone implant for a femur. In contrast to the third interface 110 of the bone implant for the tibia, the third interface 110 of the bone implant for the femur has none of the protrusions described for the bone implant for the tibia.

FIG. 3b shows a section A-A through the augment 102 for the bone implant for the femur at a section line labeled A-A in FIG. 3b.

FIG. 4 shows a schematic, perspective view of the augment 102 for the bone implant for the femur. In the example, the third interface 110 is designed to be arranged next to a fastening element for a plateau of the bone implant for the femur.

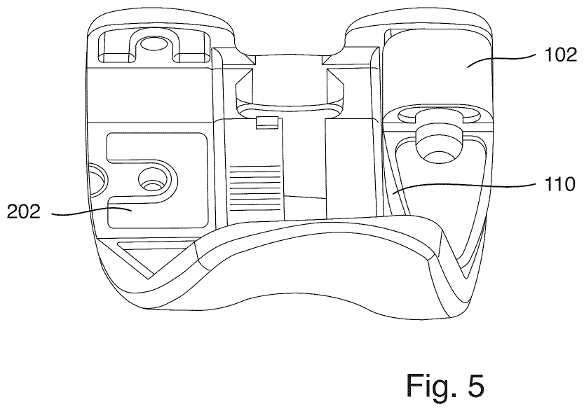

FIG. 5 shows a schematic, perspective view of the augment 102 and at least portion of the bone implant for the femur.

The plateau 202 can be positioned on a femur that has been prepared for this purpose with a horizontal saw cut. A portion of the plateau 202 is designed for anchoring the bone implant in a prepared medullary cavity of the femur.

The third interface 110 is arranged next to the portion of the plateau 202.

The tibia and femur are examples of bones for which the augment 102 can be used.

The augment 102 is designed to fill a space between the plateau 202 and the bone. The distance is, for example, a distance between a first horizontal saw cut and a second horizontal saw cut set back distally from it.

In the example, the plateau 202 and the augment 102 are connected to each other at the first interface 106. In the example, the plateau 202 is partially connected directly to the bone. In the example, the augment 102 is connected to the bone at the second interface 108.

In the example, the plateau 202 and the augment 102 are designed to be connected to the bone by means of bone cement. In the example, the second interface 108 and/or the third interface 110 have a rough surface for contact with the bone cement. The second interface 108 and/or the third interface 110 preferably have the rough surface in cement pockets formed on the surface of the augment 102, in particular in the gap-shaped cement pocket.

In the example, the bone cement is arranged in the undercut. Bone cement in the undercut further increases the fixation of the bone implant fitted with the augment 102. The augments 102 are easily accessible during a revision cut. The cement fixation by the bone cement in the undercut can be easily removed with the revision incision.

A set of augments 102 can be provided for a surgeon.

For example, the set of augments 102 includes at least two of the described augments 102, wherein the body 104 of the at least two augments 102 extend between the first interface 106 and the second interface 108 at different heights.

The set of augments 102 comprises, for example, at least two of the described augments 102, wherein the third interface 110 for receiving differently sized fastening elements 206 is configured.

The set of augments 102 comprises, for example, at least two of the described augments 102, wherein the undercuts differ in shape or angle.

In one example, the set comprises a first augment 102 and a second augment 102, which are designed as described for the bone implant. The first augment 102 and the second augment 102 are designed to be arranged against each other. The first interface 106 for fastening the first augment 102 to the plateau 202 of the bone implant and the second interface 108 for fastening the second augment 102 to the bone are designed to connect the first augment 102 and the second augment 102. This means that the augments 102 can be stacked in a pile. This allows different differences to be filled in.

It may be provided that the second augment 102 is formed without an undercut at its third interface 110 to the fastening element 206. The cement fixation is provided, for example, in an augment 102 of the pile arranged closest to the bone.

1. An augment for a bone implant, the augment having a body that comprises:
a first interface for fastening the augment to a plateau of the bone implant;
a second interface for fastening the augment to a bone; and
a third interface to a fastening element for anchoring the bone implant in a medullary cavity of the bone,
the third interface extending between the first interface and the second interface, and
the body having an undercut at the third interface.

2. The augment according to claim 1, wherein the bone implant is for a tibia or a femur.

3. The augment according to claim 1, wherein the undercut comprises a conical or concave course of the body from the second interface to the first interface.

4. The augment according to claim 1, wherein:
the body extends between the first interface and the second interface at a first height, and
the undercut extends between the first interface and the second interface at a second height that is smaller than the first height.

5. The augment according to claim 1, wherein:
the second interface extends in a plane,
the undercut comprises a wall of the body that is arranged at least in sections at an angle to the plane.

6. The augment according to claim 5, wherein the angle is between 89° and 45° to the plane.

7. The augment according to claim 6, wherein the angle is between 89° and 79° to the plane.

8. The augment according to claim 1, wherein the body tapers outside the third interface from the first interface to the second interface.

9. The augment according to claim 1, wherein:
the second interface extends in a plane, and
the body extends outside the third interface from the first interface to the second interface at an angle to the plane.

10. The augment according to claim 9, wherein the angle is 90° to the plane.

11. The augment according to claim 1, wherein the body tapers from the first interface to the second interface.

12. The augment according to claim 1, wherein:
the body comprises a circumferential area extending between the first interface and the second interface, and
the circumferential area comprises the third interface to the fastening element.

13. The augment according to claim 12, wherein the circumferential area has a rough surface at least in sections.

14. The augment according to claim 12, wherein an area outside the third interface has a smooth surface.

15. The augment according to claim 1, wherein:
the third interface comprises a portion of a circumference of the body extending between the first interface and the second interface, and
the undercut extends along the portion of the circumference of the body.

16. The augment according to claim 15, wherein the undercut extends over all of the portion of the circumference.

17. A set of augments comprising at least two augments according to claim 1, wherein:
the at least two augments comprise a first augment and a second augment,
the body of the first augment extends between the first interface and the second interface of the first augment at a first height, and
the body of the second augment extends between the first interface and the second interface of the second augment at a second height that is different than the first height.

18. A set of augments comprising at least two augments according to claim 1, wherein:
the at least two augments comprise a first augment and a second augment,
the third interface of the first augment is configured to fasten to a first fastening element of a first size, and
the third interface of the first augment is configured to fasten to a second fastening element of a second size that is different than the first size.

19. A set of augments comprising at least two augments according to claim 1, wherein:
the at least two augments comprise a first augment and a second augment,
the first augment and the second augment are arrangeable against each other, and
the first interface of the first augment is connectable to the second interface of the second augment to connect the first augment to the second augment.

20. The set of augments according to claim 19, wherein the third interface of the second augment is formed without an undercut.
